SEQUENCE LISTING (5 sequences)

SEQ ID NO: 1 - DNA, 29 bp, Artificial Sequence
  forward primer specific for sTNFR
atggcgcccgtcgccgtctgggccgcgct

SEQ ID NO: 2 - DNA, 34 bp, Artificial Sequence
  reverse primer specific for sTNFR
agtactcccttcagctggggggctggggcccatt

SEQ ID NO: 3 - DNA, 33 bp, Artificial Sequence
  forward primer specific for the Fc region of human IgG1
agtactggcgacgagcccaaatcttgtgacaaa

SEQ ID NO: 4 - DNA, 21 bp, Artificial Sequence
  reverse primer specific for the Fc region of human IgG1
tcatttacccggggacaggga

SEQ ID NO: 5 - DNA, 1470 bp, Artificial Sequence
  sTNFRFc fusion gene of soluble p75 TNFR and Fc portion of human IgG1
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagtactggcgacgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

(19) United States
(12) Patent Application Publication
(10) Pub. No.: US 2004/0082533 A1
(43) Pub. Date: Apr. 29, 2004

(54) Electro-gene therapy of arthritis by using an expression plasmid encoding the soluble p75 tumor necrosis factor receptor-Fc fusion protein

(76) Inventors: Jong-Mook Kim, Seoul (KR); Seong-Hyun Ho, Incheon (KR); Woong Hahn, Seoul (KR); Seung-Shin Yu, Seoul (KR); Sunyoung Kim, Seoul (KR)

Correspondence Address: Anderson Kill & Olick, P.C., 1251 Avenue of the Americas, New York, NY, 10020, US

(21) Appl. No.: 10/637,864

(22) Filed: Aug. 8, 2003

Related U.S. Application Data
(60) Provisional application No. 60/402,399, filed on Aug. 9, 2002.

(51) Int. Cl. A61N 1/30; A61K 48/00

(52) U.S. Cl. 514/44; 604/20

(57) ABSTRACT

The electroporation-mediated delivery of plasmid containing cDNA for soluble p75 TNF (tumor necrosis factor) receptor linked to the Fc portion of human IgG1 (sTNFR:Fc) can be effectively used for the treatment of arthritis in a mammal.

### CROSS REFERENCE TO RELATED APPLICATION

This application is a continuation-in-part (CIP) application of U.S. Ser. No. 60/402,399, which was filed on Aug. 9, 2002.

### FIELD OF THE INVENTION

The present invention relates to a pharmaceutical composition for electro-gene therapy of arthritis which comprises a plasmid DNA encoding soluble p75 TNF (tumor necrosis factor) receptor linked to the Fc portion of human IgG1 (sTNFR:Fc); and a method for electro-gene therapy of arthritis by injecting same into the muscles using in vivo electroporation.

### BACKGROUND OF THE INVENTION

Rheumatoid arthritis (RA) is a chronic disease characterized by inflammation of the joints with concomitant destruction of both cartilage and bone (Kaklamanis, P. M., Clin. Rheumatol. 11: 41-47, 1992). Although the causes of RA are not fully understood, various experimental and clinical studies suggest that proinflammatory cytokines, particularly TNF-α, play an important role in RA pathogenesis (Deleuran, B. W. et al., Arthritis Rheum. 35: 1170-1178, 1992; Arend, W. P. et al., Arthritis Rheum. 38:151-160, 1995; Brennan, F. M. et al., Curr. Opin. Immunol. 4: 754-759, 1992; Thorbecke, G. J. et al., Proc. Natl. Acad. Sci. USA 89: 7375-7379, 1992; Joosten, L. A. et al., Arthritis Rheum. 39: 797-809, 1996). TNF concentrations are elevated in the synovial fluid of persons with active rheumatoid arthritis (Chu, C. Q. et al., Arthritis Rheum. 34: 1125-1132, 1991; Saxne, T. et al., Arthritis Rheum. 31: 1041-1045, 1988) and increased plasma levels of TNF are associated with joint pain (Beckham, J. C. et al., J Clin. Immunol. 12: 353-361, 1992).

There are two distinct type cell-surface TNF receptors (TNFRs), designated p55 and p75 (Smith, C. A. et al., Science 248: 1019-1023, 1990; Loetscher H. et al., Cell 61: 351-359, 1990). Soluble, truncated versions of membrane TNFRs (sTNFR), consisting of only the extracellular, ligand-binding domain, are present in body fluids and are thought to be involved in regulating TNF activity (Engelmann, H. et al., J. Biol. Chem. 264: 11974-11980, 1989; Olsson, I. et al., Eur. J Haematol. 41: 270-275, 1989). Recombinant sTNFR:Fc fusion proteins, which are engineered sTNFRs linked to the Fc portion of immunoglobulin G1 (IgG1), have been developed for therapeutic neutralization of TNF (Mohler, K. M. et al., J. Immunol. 151: 1548-1561, 1993; Evans, T. J. et al., J. Exp. Med. 180: 2173-2179, 1994). Several experimental and clinical studies demonstrated that the p75 TNFR:Fc fusion protein is effective in RA while the p55 TNFR:Fc fusion protein worked, but to a lesser extent (Wooley, P. H. et al., J. Immunol. 151: 6602-6607, 1993; Moreland, L. W. et al., N. Engl. J Med. 337: 141-147, 1997; Hasler, F. et al., Arthritis Rheum. 39: S:243, 1996).

Effective control of autoimmune arthritis requires prolonged neutralization of proinflammatory mediators, and gene therapy offers several potentially unique advantages over previous protein therapies. With the recent advances in gene therapy, the TNFR gene has been delivered by retrovirus-based and adenovirus-based vectors intraarticularly or systemically to achieve anti-inflammatory effects with varying degrees of success (Ghivizzani, S. C. et al., Proc. Natl. Acad. Sci. USA 4613-4618, 1998; Mageed, R. A. et al., Gene Ther. 5: 1584-1592, 1998; Le, C. H. et al., Arthritis Rheum. 40: 1662-1669, 1997; Quattrocchi, E. et al., J. Immunol. 163: 1000-1009, 1999).

Among various viral and non-viral techniques for gene transfer in vivo, the direct injection of plasmid DNA into the muscle is probably the simplest, most inexpensive and safest method (Nishikawa, M. et al., Hum. Gene Ther. 12: 861-870, 2001). Since plasmid DNA injection followed by in vivo electroporation has been shown to be effective for introducing DNA into murine muscles, the present inventors have therefore endeavored to develop a method for electro-gene therapy of arthritis by injecting a plasmid DNA encoding the human sTNFR:Fc gene to the muscles using in vivo electroporation.

### SUMMARY OF THE INVENTION

Accordingly, it is an object of the present invention to provide a pharmaceutical composition for treating arthritis in a mammal.

Another object of the present invention is to provide a method for treating arthritis in a mammal.

In accordance with one aspect of the present invention, there is provided a pharmaceutical composition for electro-gene therapy of arthritis in a mammal, which comprises a plasmid DNA encoding soluble p75 TNF (tumor necrosis factor) receptor linked to the Fc portion of human IgG1 (sTNFR:Fc).

In accordance with another aspect of the present invention, there is provided a method for electro-gene therapy of arthritis in a mammal, which comprises injecting an effective amount of the DNA encoding sTNFR:Fc to the muscles via in vivo electroporation.

### BRIEF DESCRIPTION OF THE DRAWINGS

The above and other objects and features of the present invention will become apparent from the following description of the invention, when taken in conjunction with the accompanying drawings, which respectively show:

FIG. 1a: the structure of pCK-sTNFR:Fc, wherein the numbers indicate the relative positions to the transcription initiation site (+1) of the major immediate early promoter (MIEP) of the human cytomegalovirus (HCMV);

| | |
|---|---|
| hatched box: MIEP of HCMV, | dotted box: exon, |
| wavy line: intron, | pA: poly A tract, |
| Kan: kanamycin resistance gene, | ColEI: E. coli origin of replication |

FIG. 1b: immunoblot analysis of the culture supernatant obtained from 293T cells transfected with pCK-sTNFR:Fc;

FIG. 1c: ELISA analysis of the culture supernatant obtained from 293T cells transfected with pCK-sTNFR:Fc (*=P<0.01 versus control);

FIG. 2a: serum levels of sTNFR:Fc 6 days after injection in DBA/1 mice treated with different amounts of sTNFR:Fc DNA (pCK-sTNFR:Fc) or vector DNA (pCK) as indicated with (+EP) or without (−EP) electroporation;

FIG. 2b: serum levels of sTNFR:Fc over time in DBA/1 mice injected with 15 μg of sTNFR:Fc DNA or vector DNA with (+EP) or without (−EP) electroporation.

FIG. 2c: serum levels of sTNFR:Fc at indicated times in NOD/SCID mice injected with 15 μg of sTNFR:Fc DNA or vector DNA with electroporation;

FIG. 2d: the sTNFR:Fc levels in the injected muscles at indicated times in DBA/1 mice injected with 15 μg of sTNFR:Fc DNA or vector DNA with electroporation;

FIG. 2e: the sTNFR:Fc levels in knee joints at indicated times in DBA/1 mice injected with 15 μg of sTNFR:Fc DNA or vector DNA with electroporation (*=P<0.01 versus vector DNA+EP);

FIG. 3: electroporation-associated damage in gastrocnemius muscles of DBA/1 mice (at least six muscles per experimental group) injected with 15 μg of sTNFR:Fc DNA (pCK-sTNFR:Fc) or vector DNA (pCK) with (+EP) or without (−EP) electroporation;

Arrows: infiltrating inflammatory cells

Original magnification: ×200

FIG. 4: time course of therapeutic effects of the electroporation-mediated delivery of pCK-sTNFR:Fc on the occurrence of arthritis in CIA (*=P<0.05 versus control [Mann-Whitney rank sum test]; ** =P<0.05 versus control [Fisher's exact test]);

FIG. 5: effects of pCK-sTNFR:Fc on synovitis in CIA, wherein (a) shows hematoxylin-eosin staining of knee joint tissues obtained from the control mice, (b), the experimental mice treated with pCK-sTNFR:Fc, and (c), the score of synovitis in the knees of the experimental mice treated with pCK-sTNFR:Fc (*=P<0.05 versus control);

| | |
|---|---|
| Original magnification: × 100 | F: femur, |
| T: tibia, | C: cartilage, |
| S: synovium, | JS: joint space |

FIG. 6: effects of pCK-sTNFR:Fc on cartilage erosion in CIA, wherein (a) shows safranin O-staining of knee joint tissues obtained from control mice, (b), the experimental mice treated with pCK-sTNFR:Fc, and (c), the erosion of cartilage in the knees of the experimental mice treated with pCK-sTNFR:Fc (*=P<0.05 versus control);

| | |
|---|---|
| Original magnification: × 100 | F: femur, |
| T: tibia, | C: cartilage, |
| S: synovium, | JS: joint space |

FIG. 7: effects of pCK-sTNFR:Fc on the level of IL-1β (a), IL-12 (b), IL-17 (c), and vWF (d) in the ankle joints of mice with CIA (*=P<0.01 versus control time).

### DETAILED DESCRIPTION OF THE INVENTION

The present invention provides a pharmaceutical composition for electro-gene therapy of arthritis in a mammal, which comprises a plasmid DNA encoding soluble p75 TNF (tumor necrosis factor) receptor linked to the Fc protein of human IgG1 (sTNFR:Fc).

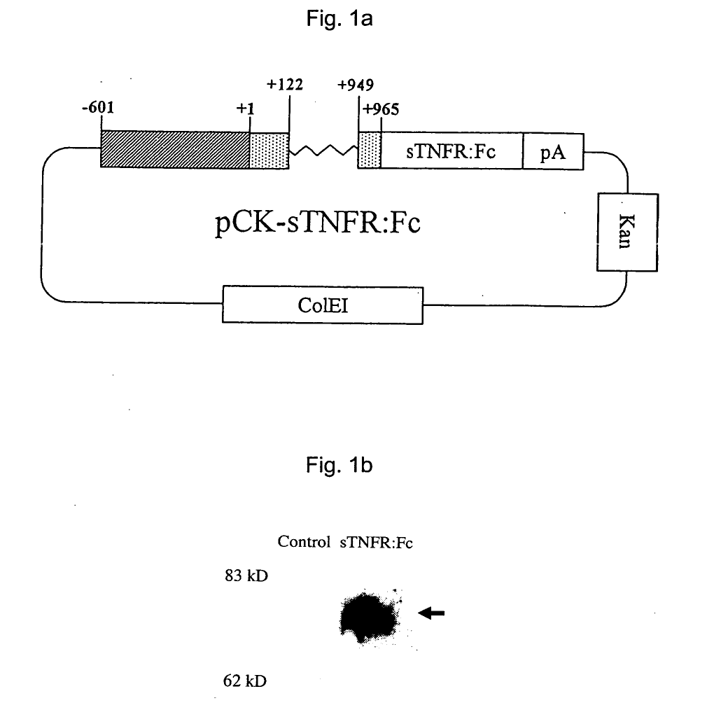

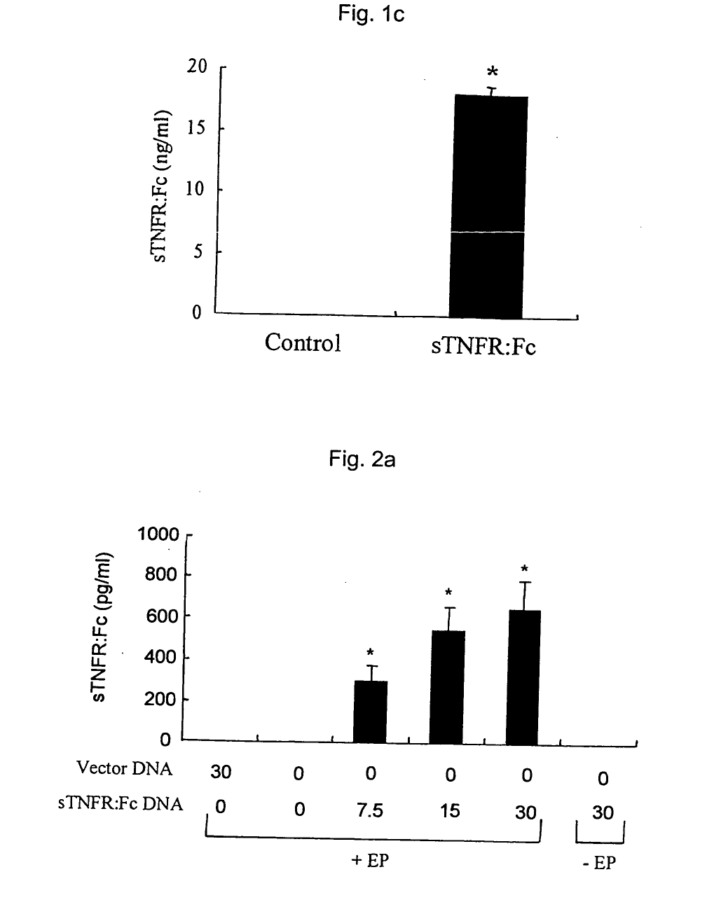

Constructed in the present invention is an expression plasmid encoding sTNFR:Fc fusion gene being which the soluble p75 TNF (tumor necrosis factor) receptor is linked to the Fc portion of human IgG1. In accordance with a preferred embodiment of the present invention, the sTNFR:Fc fusion gene of SEQ ID NO: 5 is inserted into pCK vector which gives a high expression level of a foreign gene (Lee, Y. et al., Biochem. Biophys. Res. Commun. 272: 230-235, 2000), which is designated “pCK-sTNFR:Fc” (see FIG. 1a).

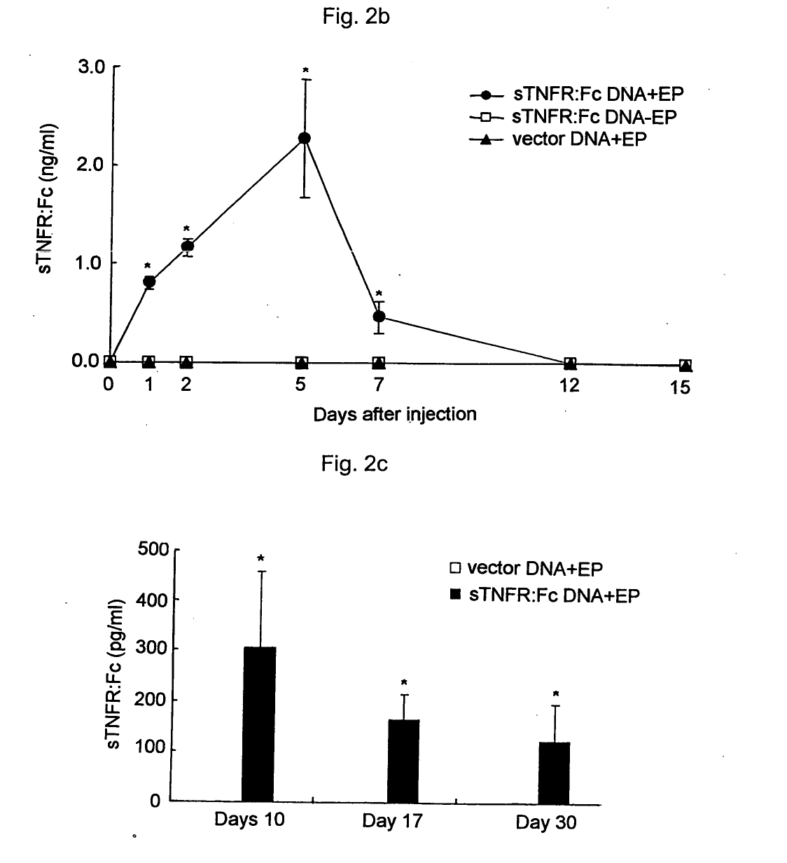

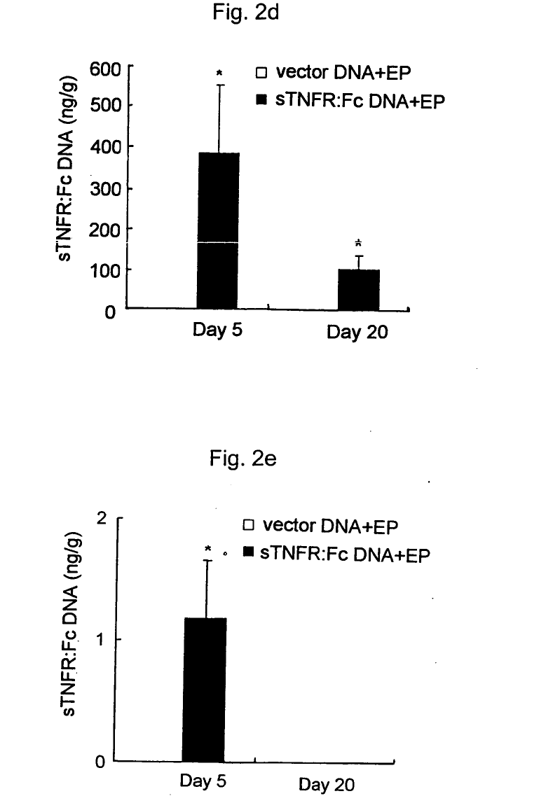

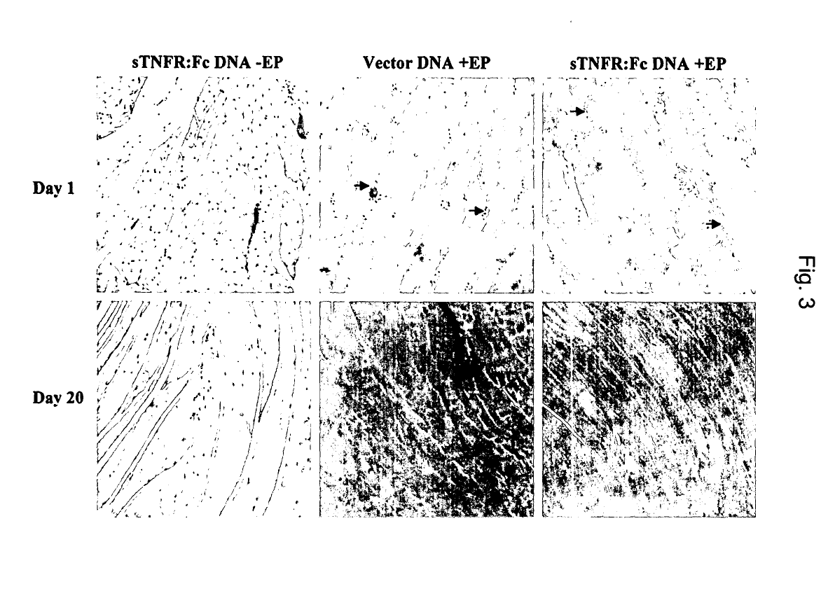

Immunoblotting of the culture supernatant 293T cells transfected with pCK-sTNFR:Fc reveals the expression of a 76-kDa protein, the expected size of human sTNFR:Fc (see FIG. 1b). A significantly high level of sTNFR:Fc is produced by 293T cells (1×10⁵) transfected with sTNFR:Fc, which showed be compared with the identical number of cells transfected with the control plasmid (see FIG. 1c). The result of examining serum sTNFR:Fc levels in DBA/1 mice injected with different amounts of DNA with or without in vivo electroporation by ELISA show significant levels of sTNFR:Fc produced by in vivo electroporation in a dose-dependent manner (see FIG. 2a), for a duration of 7 days after injection of pCK-sTNFR:Fc (see FIG. 2b). For contrast significant levels of pCK-sTNFR:Fc are detected in the sera of NOD/SCID mice even 30 days after DNA treatment (see FIG. 2c), suggesting the possible role of immune response in a relatively short period of sTNFR:Fc expression in immunocompetent mice. Further, significant levels of sTNFR:Fc are found in the muscles and knee joints, which showed be compared with the control (see FIGS. 2d and 2e). As a result of histological examination to assess electroporation-mediated damage, there was no significant difference in the degree of inflammation between the vector DNA- and the sTNFR:Fc DNA-treated group (see FIG. 3). These results clearly indicate that in vivo electroporation is a highly efficient method for the systematic delivery of sTNFR:Fc.

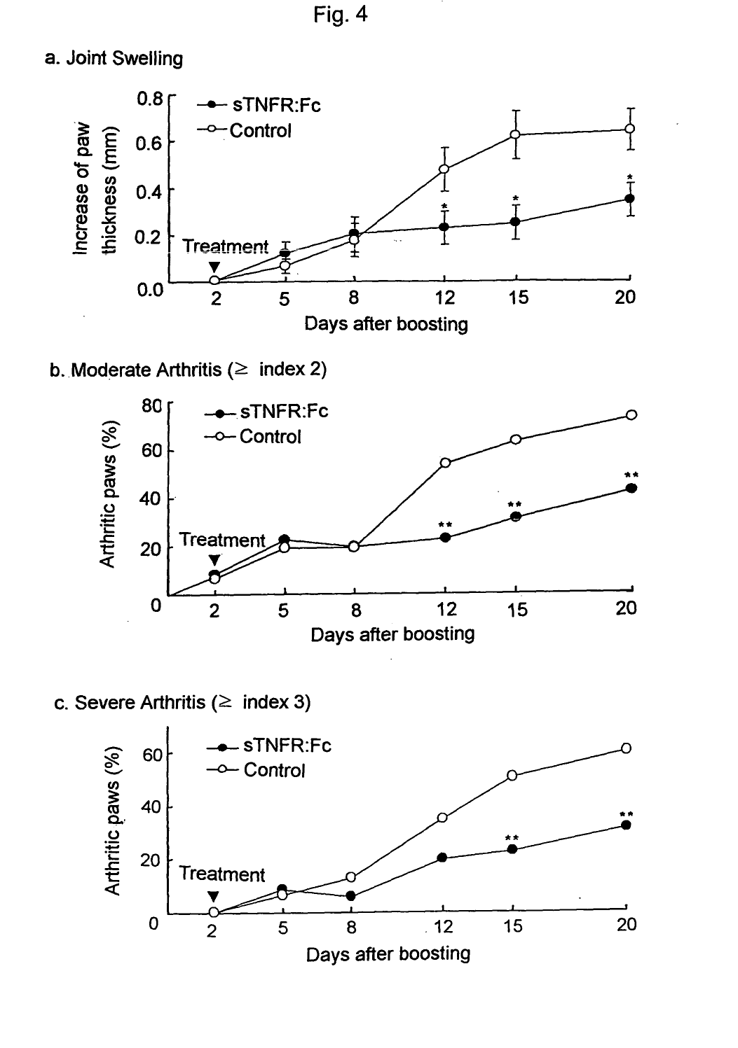

Macroscopic examination to assess the incidence of arthritis in the paws has revealed that electroporation-mediated transfer of pCK-sTNFR:Fc can efficiently reduce the incidence of moderate to severe CIA and beneficial effects of a single electroporation-mediated gene transfer last for a minimum of 18 days following treatment (see FIGS. 4a to 4c).

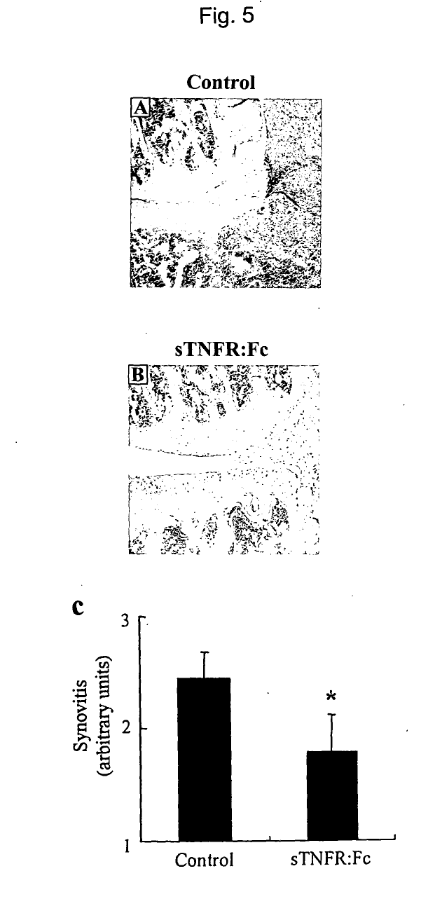

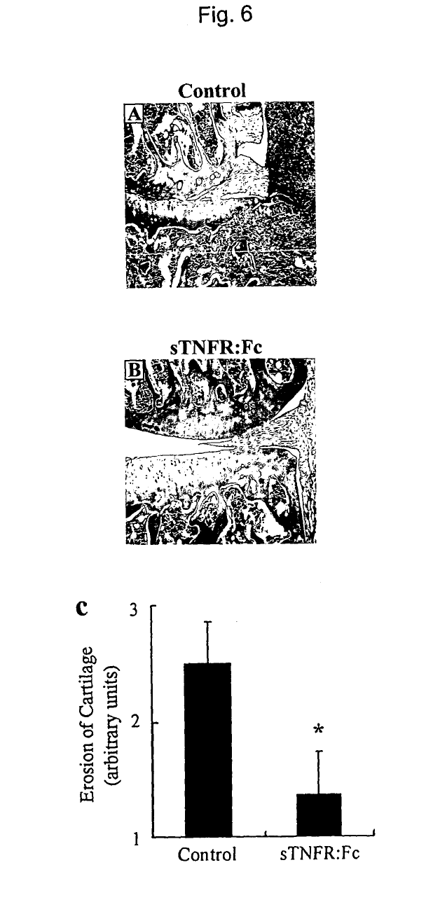

Histological analysis showed that synovial proliferation and inflammatory cell infiltration are significantly suppressed (hematoxylin/eosin staining) and that the proteoglycan in the cartilage is well-preserved (safranin O-staining) in the joints of mice treated with sTNFR:Fc, but not in the joints treated with control plasmid DNA (see FIGS. 5 and 6). These results has demonstrated that electroporation-mediated delivery of pCK-sTNFR:Fc efficiently reduces the degree of histopathologic changes in the knee joints of CIA mice.

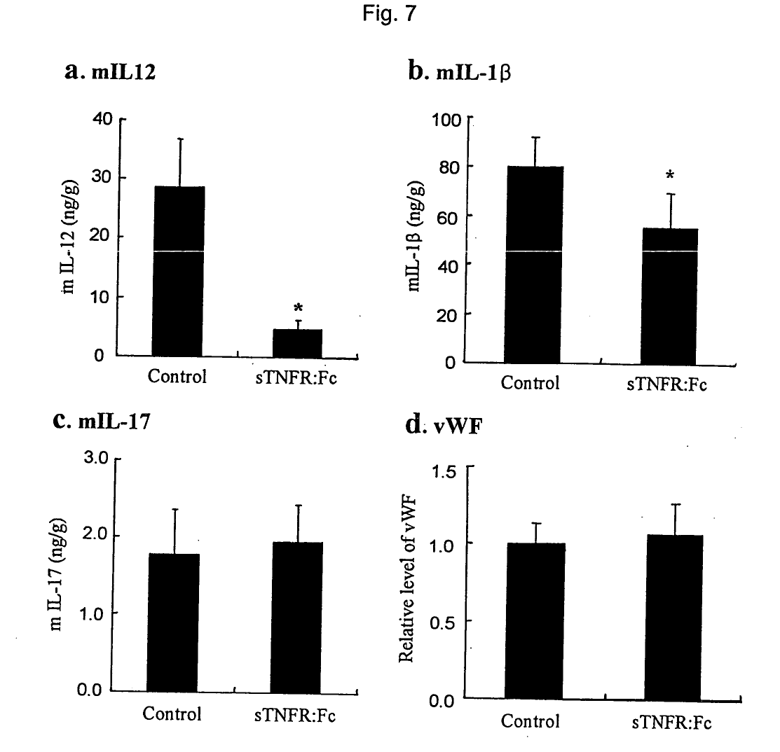

As a result of examining the effects of pCK-sTNFR:Fc on the levels of IL-1β, IL-12, IL-17 and vWF in the ankle joints of mice with CIA, the production of IL-1β and IL-12 are lower in the sTNFR:Fc-treated mice relative to the levels seen in the control vector-treated group, while the levels of IL-17 and vWF remain unchanged (see FIG. 7). These results suggested that delivery of sTNFR:Fc DNA by electroporation can efficiently reduce the incidence of CIA by modulating the levels of inflammatory cytokines such as IL-1β and IL-12.

In the inventive anti-TNF gene therapy, the arthritis can effectively be treated by administering an expression plasmid encoding sTNFR:Fc via in vivo electroporation.

The present invention demonstrates that delivery of plasmid DNA containing cDNA for human sTNFR:Fc by in vivo electroporation can reduce the incidence and severity of murine collagen-induced arthritis and that such beneficial effects last for 18 days after a single treatment. Further, the electrotransfer of sTNFR:Fc DNA reduces the levels of IL-1β and IL-12 in the joints of treated CIA mice. It has been reported that IL-1β and IL-12 each plays an important role in the pathogenesis of arthritis (Arend, W. P. et al., Arthritis Rheum. 38: 151-160, 1995; Dayer, J. M., Joint Bone Spine 69:123-132, 2002; Arner, E. C. et al., Arthritis Rheum. 32: 288-297, 1989; Joosten, L. A. et al., J. Immunol. 159: 4094-4102, 1997; Malfait, A. M. et al., Clin. Exp. Immunol. 111: 377-383, 1998). Therefore, one of the possible mechanisms involved in the suppression of arthritis by sTNFR:Fc is the inhibition of TNF-α-induced production of IL-1β and IL-12. The inhibitory effect of sTNFR:Fc on the level of IL-12 also suggests that sTNFR:Fc may down-regulate Th1 activity, since IL-12 is known to play a pivotal role in promoting the differentiation of Th1 responses and inducing IFNγ production (Triantaphyllopoulos, K. A. et al., Arthritis Rheum. 42: 90-99, 1999).

The present invention shows that a significantly high level of sTNFR:Fc in sera can be maintained for 7 days by a single in vivo electroporation procedure, the duration being significantly shorter than other cases using a mouse erythropoietin gene. One possible explanation for the limited duration of human sTNFR:Fc expression is related to the immune response to the human sTNFR:Fc protein in mice. This possibility is supported by the observation that the expression of human sTNFR:Fc lasts for 30 days in NOD/SCID mice under identical conditions. Therefore, a longer period of sustained sTNFR:Fc expression can be expected in the human system.

The present invention demonstrates for the first time that electroporation-mediated delivery of a plasmid containing cDNA for sTNFR:Fc can be used to modulate the disease process in an animal arthritis model. Therefore, the inventive expression plasmid encoding sTNFR:Fc may help to develop the clinically relevant protocol for electroporation-based gene delivery strategy for the treatment of human RA.

Accordingly, the present invention provides a pharmaceutical composition for treating arthritis by in vivo electroporation-mediated gene transfer of sTNFR:Fc, which comprises the expression plasmid encoding sTNFR:Fc as an effective ingredient, in combination with pharmaceutically acceptable excipients, carriers or diluents.

The inventive pharmaceutical formulation may be prepared in accordance with any one of the conventional procedures. In preparing the formulation, the effective ingredient is preferably admixed or diluted with a carrier. Examples of suitable carriers, excipients, or diluents are lactose, dextrose, sucrose, sorbitol, mannitol, starches, gum acacia, alginates, gelatin, calcium phosphate, calcium silicate, cellulose, methylcellulose, microcrystalline cellulose, polyvinylpyrrolidone, water, methylhydroxybenzoates, propylhydroxybenzoates, talc, magnesium stearate and mineral oil. The formulation may additionally include fillers, anti-agglutinating agents, lubricating agents, wetting agents, flavoring agents, emulsifiers, preservatives and the like. The composition of the invention may be formulated so as to provide a quick, sustained or delayed release of the active ingredient after it is administrated to a patient, by employing any one of the procedures well known in the art.

The pharmaceutical formulation of the present invention can be administered via intramuscular introduction with in vivo electroporation.

Further, the present invention provides a method for electro-gene therapy of arthritis in a mammal, which comprises administering an effective amount of the expression plasmid encoding sTNFR:Fc in the muscles via in vivo electroporation.

In contrast to protein therapy, gene therapy has the advantage of relatively long-lasting expression at low levels, and therefore allows for reduced frequency of administration. This consideration is especially important in chronic diseases, such as RA, which may require long-term therapy. The present invention indicated that beneficial effects lasted at least 18 days per single injection of plasmid DNA encoding the cDNA of sTNFR:Fc followed in vivo electroporation. These observations suggested that gene therapy for RA using the delivery of a plasmid vector by electroporation might be a therapeutically plausible form of RA treatment. This plasmid DNA transfer method has several advantages over viral vectors. A large quantity of highly purified plasmid DNA can be readily obtained at a relatively low cost, and gene transfer can be repeated without apparent immunological responses to the plasmid DNA vector. Furthermore, quality control of DNA production, an important step on an industrial scale, is expected to be much less complicated than other viral vectors.

For treating a human patient, a typical daily dose of the inventive expression plasmid encoding sTNFR:Fc may range from about 0.005 to 50 mg/kg body weight, preferably 0.05 to 5 mg/kg body weight, and can be administered in a single dose or in divided doses. However, it should be understood that the amount of the active ingredient actually administered ought to be determined in light of various relevant factors including the condition to be treated, the chosen route of administration, the age, sex and body weight of the individual patient, and the severity of the patient's symptom; and, therefore, the above dose should not be intended to limit the scope of the invention in any way.

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usage and conditions.

### REFERENCE EXAMPLE 1

Cloning of Human sTNFR:Fc and Construction of Expression Vector

cDNAs encoding the human sTNFR and Fc regions of human IgG1 were cloned from total RNA prepared from human peripheral blood lymphocytes by reverse transcription-polymerase chain reaction (RT-PCR), respectively. PCR primers were: SEQ ID NOs: 1 and 2 for sTNFR; SEQ ID NOs; 3 and 4 for the Fc region of human IgG1. The amplified cDNAs were initially cloned into the pGEM-T easy plasmid (Promega, Wis., USA), to obtain pGEM-sTNFR and pGEM-Fc, respectively. Following sequence confirmation, the ScaI fragment of the pGEM-sTNFR, which contains the sTNFR cDNA, was cloned into the ScaI site of the pGEM-Fc, to obtain pGEM-sTNFR:Fc. Subsequently, the DNA fragment encoding sTNFR:Fc was cloned into the EcoRI site of the mammalian expression vector pCK (Lee, Y. et al., Biochem. Biophys. Res. Commun. 272: 230-235, 2000), to prepare pCK-sTNFR:Fc. The pCK-sTNFR:Fc plasmid was purified using an EndoFree plasmid Maxi prep kit (Qiagen, Valencia, Calif., USA), dissolved in 0.9% NaCl, diluted to 4 μg/μl and stored at −20° C. prior to use.

### REFERENCE EXAMPLE 2

SDS-PAGE and Western Blotting

pCK-sTNFR:Fc and pCK were transfected into 293T cells with FuGENE6 (Roche Diagnostics, Germany), respectively. Two days after transfection, each of the culture supernatants was mixed with a one-third volume of sodium dodecyl sulfate (SDS) sample buffer (75 mM Tris-HCl [pH 6.8], 6% SDS, 15% glycerol, 15% 2-mercaptoenthanol, and 0.015% bromophenol blue), heated at 98° C. for 5 min, and subjected to SDS-polyacrylamide gel electrophoresis (PAGE) on a 10% polyacrylamide gel. After electrophoresis, the sample was transferred onto a polyvinylidene difluoride membrane (Millipore, Bedford, Mass.). The membrane was incubated at room temperature for 3 hours with horseradish peroxidase (HRP)-conjugated anti-human IgG (Pierce, Rockford, Ill.), washed, and processed for autoradiography using chemiluminescence techniques (ECL kit: Amersham, Ill.), according to the manufacturer's instructions.

### REFERENCE EXAMPLE 3

Intramuscular DNA Injection and Electroporation

Mice were anaesthetized with ketamine (1.35 mg/mouse)/xylazine (66 μg/mouse). Aliquots of 30 μl of plasmid DNA (pCK-sTNFR:Fc or control pCK) at 0.25, 0.5, or 1 μg/μl in 0.45% NaCl were injected into the gastrocnemius muscle of the left hind leg (total amount of DNA was 7.5, 15, or 30 μg per mouse). Ninety units of type VI-S hyaluronidase obtained from bovine testes (Sigma, St. Louis, Mo.) were resuspended in 50 μl of sterile saline solution and injected 10 min prior to electroporation as previously described (Mennuni, C. et al., Hum. Gene Ther. 13:355-365, 2002). Commercially available caliper electrodes (model 383, BTX, San Diego, Calif.) were used for electroporation. The caliper electrodes were applied to the shaved skin on either side of the marked DNA injection point, and the calipers were closed to a gap of 5 mm, so that electrical contact with the skin was maximized. Consecutively square-wave electrical pulses were administered 8 times using an ECM830 pulse generator (BTX, San Diego, Calif.) at 200 V/cm and a rate of one pulse/sec., with each pulse being 20 msec. in duration.

### REFERENCE EXAMPLE 4

Induction of CIA and Treatment Protocol

DBA/1 mice (Charles River, Mass., USA), aged 6-7 weeks at the start of experiments, were immunized intradermally at the base of the tail with bovine type II collagen (100 μg; Chondrex, Wash.) emulsified in Freund's complete adjuvant (GIBCO BRL, NY) (Kim, J. M. et al., Arthritis Rheum. 46: 793-801, 2002). On day 21, the animals were boosted with an intradermal injection of 100 μg type II collagen emulsified in Freund's incomplete adjuvant (GIBCO BRL, NY). Two days after the boosting, the mice were divided into 2 groups and individually treated with either 15 μg of plasmid DNA containing cDNA for sTNFR:Fc or control plasmid. For each mouse, one site in the gastrocnemius muscle of the left hind leg received direct injections with plasmid DNA with the use of a 1 ml syringe at a 27-gauge needle (15 μg/30 μl for each mouse), followed by in vivo electroporation.

### REFERENCE EXAMPLE 5

Macroscopic Scoring of CIA

Paws were individually scored using a macroscopic system in a scale of 0 to 4, as previously described, with a maximum score of 4 for each paw: 0=normal; 1=detectable arthritis with erythma; 2=significant swelling and redness; 3=severe swelling and redness from joint to digit; and, 4=maximal swelling and deformity with ankylosis (Kim, S. H. et al., J. Immunol. 166: 3499-3505, 2001). The thickness of each paw was also measured using a spring-load caliper. Such scoring of arthritic index and measuring the paw thickness were done by 2 independent observers who were not informed of the experimental groups.

### REFERENCE EXAMPLE 6

Histologic Processing and Analysis of Knee Joints

Knee joints were dissected, fixed in 10% phosphate-buffered formalin for 2 days, decalcified in 10% EDTA for 7 days, and then embedded in paraffin. Standard frontal sections of 7 μm were prepared, stained with either hematoxylin/eosin or Safranin O/fast green. Histopathological changes were scored using the following parameters. The severity of synovitis (synovial proliferation and inflammatory cell infiltration) was scored using a four-point scale (0-3, where 0 is normal and 3, severe) (Bessis, N. et al., J Gene Med. 4: 300-307, 2002). Cartilage destruction was separately graded on a scale from 0 to 4 for each joint, where 0=normal, 1=dead chondrocyte, 2=local destruction of superficial chodrocyte, 3=multifocal destruction of chondrocyte/subchondral bone, and 4=complete destruction of chondrocyte and massive destruction of subcondral bone. The scoring was performed on decoded slides by two non-informed observers, as previously described (Lubberts, E. et al., J. Immunol. 163: 4546-4556, 1999).

### REFERENCE EXAMPLE 7

Measurement of Cytokine Levels in Mouse Serum, Joint and Muscle

The levels of human sTNFR:Fc in sera, joints and the injected muscles, and the levels of murine IL-1β, murine IL-12, murine IL-17 and von Willebrand factor (vWF) in ankles were respectively measured using commercially available ELISAs for human TNFR (R&D Systems, Minneapolis, Minn., USA), mIL-1β (R & D Systems), mIL-12 (R & D Systems), mIL-17 (R & D Systems), and human vWF (Asserachrom vWF kit; Roche, Tokyo, Japan), according to the manufacturer's instructions. Briefly, the injected muscles were excised and homogenized in a lysis buffer (25 mM Tris-HCl [pH 7.4], 50 mM NaCl, 0.5% Na-deoxycholate, 2% NP-40, 0.2% SDS, protease inhibitors). In the case of joint tissues, whole mice knees and ankles were snap frozen in liquid nitrogen and were ground into powder with a pestle, then lysed with the lysis buffer. The supernatants containing the total protein were used to measure cytokine levels. In the case of serum, it was directly subjected to TNFR assays without any pretreatment. Levels of vWF were expressed in percentage based on assigning that of a human plasma calibration standard a value of 100% (Kim, J. M. et al., Arthritis Rheum. 46: 793-801, 2002). The levels of cytokines were normalized to the total amount of protein prepared from tissue lysates, as measured by way of a DC protein assay kit (Bio-Rad Laboratories, Hercules, Calif.).

### REFERENCE EXAMPLE 8

Statistical Analysis

Differences between experimental groups were tested using the Mann-Whitney rank sum test, unless stated otherwise. P values less than 0.05 were considered significant.

### EXAMPLE 1

In Vivo Expression of sTNFR:Fc by Electroporation

Used as a plasmid expression vector for intramuscular gene therapy was pCK, which has been shown to drive a high level of gene expression in the skeletal and cardiac muscles of mice (Lee, Y et al., Biochem. Biophys. Res. Commun. 272: 230-235, 2000; Lim, B. K. et al., Circulation 105: 1278-1281, 2002). The human sTNFR:Fc coding sequence was cloned to pCK, to obtain in pCK-sTNFR:Fc. (FIG. 1a). Immunoblotting of the culture supernatant from 293T cells transfected with pCK-sTNFR:Fc was performed as described in Reference Example 2. As a result, anti-human IgG antibody detected a protein of 76-kDa, which is the expected size for human sTNFR:Fc (FIG. 1b). The level of sTNFR:Fc in the culture supernatant was examined by ELISA. As shown in FIG. 1c, 18.1 ng/ml of sTNFR:Fc was produced from 1×10⁵ of 293T cells transfected with pCK-sTNFR:Fc, while less than 10 pg/ml was detected in the same number of cells transfected with the control plasmid (pCK).

To determine whether the electroporation-mediated transfer of pCK-sTNFR:Fc produced physiologically significant levels of protein, one site of the left gastrocnemius muscle of each DBA/1 mouse was injected with different amounts of DNA with or without in vivo electroporation. As a control, the same amount of vector DNA (pCK) was used. Six days after DNA injection, the serum concentrations of sTNFR:Fc were determined by ELISA. As shown in FIG. 2a, significant levels of sTNFR:Fc were produced by in vivo electroporation in a dose dependent manner, while the sTNFR:Fc levels of the control mice similarly treated with the vector DNA or sTNFR:Fc DNA without electroporation were less than 1 pg/ml, the detection threshold of the assay. Based on this result, 15 μg/per mouse of DNA was chosen for later experiments.

Investigated next was how long the sTNFR:Fc could be expressed by a single in vivo electroporation. 15 μg/per mouse of DNA was injected to the gastrocnemius muscle of mice with or without in vivo electroporation. As a control, the same amount of vector DNA was injected into another group of mice with in vivo electroporation. The serum concentrations of sTNFR:Fc were examined by ELISA at appropriate times after electroporation. As shown in FIG. 2b, significant levels (higher than 100 pg/ml) of serum sTNFR:Fc were detected for the duration of 7 days after injection of pCK-sTNFR:Fc (P<0.01, compared with pCK). The serum sTNFR:Fc concentration rapidly increased immediately after electroporation, reaching its peak at 2.3 ng/ml on day 5, while that of the control mice similarly treated with the empty pCK vector was less than 1 pg/ml, the detection threshold of the assay. The level of serum sTNFR:Fc in the control mice treated with sTNFR:Fc without electroporation was less than 1 pg/ml. Further, similar experiments using NOD/SCID mice was performed. In contrast to the above results, significant levels of sTNFR:Fc were detected in the sera of NOD/SCID mice even 30 days after DNA treatment (FIG. 2c), suggesting the possible role of immune response in a relatively short period of sTNFR:Fc expression in immunocompetent mice.

Furthermore, the levels of sTNFR:Fc were examined in the knee joints and the injected areas of muscles. As seen in FIG. 2d, significant levels of sTNFR:Fc were detected in the muscles even at 20 days after in vivo electroporation. In the knee joints, the level of sTNFR:Fc was low but still significantly higher in sTNFR:Fc DNA-treated mice as compared with control DNA-treated mice 5 days after DNA treatment (FIG. 2e). Data are presented in the form of the mean± SEM of sTNFR:Fc measured in ten samples (*=P<0.01 versus vector DNA+EP).

In addition, the effects of in vivo electroporation on the local inflammation were analyzed in the injected areas of muscles by histological examination. Gastrocnemius muscles of DBA/1 mice (at least six muscles per experimental group) were injected with 15 μg of sTNFR:Fc DNA (pCK-sTNFR:Fc) or vector DNA (pCK) with (+EP) or without (−EP) electroporation. One day or 20 days after treatment, muscles were harvested, fixed in 10% phosphate-buffered formalin, and embedded in paraffin. Sections (5 μm) were cut and stained with hematoxylin and eosin. As a result, increased inflammation was observed in both the vector DNA-treated group and the sTNFR:Fc DNA-treated group one day after in vivo electroporation, but these phenomena almost disappeared 20 days after DNA treatment (FIG. 3). There was no significant difference in the degree of inflammation between the control DNA- and the sTNFR:Fc DNA-treated group. These results clearly indicate that in vivo electroporation is a highly efficient method for the systematic delivery of sTNFR:Fc.

### EXAMPLE 2

Time Course of Therapeutic Effects of the Electroporation-Mediated Delivery of pCK-sTNFR:Fc on Arthritis in CIA

Whether the in vivo electroporation-mediated gene transfer or sTNFR:Fc could be used to prevent experimental arthritis was tested. DBA/1 mice were immunized with bovine type II collagen, then 21 days after the initial immunization the mice were boosted with the same antigen. Two days after the boosting, the mice were divided into 2 groups. 15 μg/per mouse of pCK-sTNFR:Fc was injected into one site in the gastrocnemius muscle of each mouse followed by in vivo electroporation in one group. As a control, the same amount of a control plasmid lacking the sTNFR:Fc coding sequence, pCK, was used for a separate group of mice.

The incidence of arthritis in the paws was assessed by macroscopic examination such as joint swelling and erythema every three or five days until day 20 following boosting. Joint swelling of the paw was evaluated by determining the increase in paw thickness and graded on a relative scale of 0-4 as described in Reference Example 5. The score ≧2 was considered as moderate arthritis and the score ≧3 was considered as severe arthritis.

As shown in FIG. 4a, the increase of paw thickness was significantly smaller in mice treated with pCK-sTNFR:Fc, as compared with that in mice treated with the control plasmid 20 days after boosting (P<0.05). Twenty days following boosting, the incidence of moderate to severe arthritis (≧index 2) was seen in 72% of the paws of those mice which had received control plasmid DNA, while it was only evident in 42% of the paws of those mice treated with pCK-sTNFR:Fc (P<0.05) (FIG. 4b). Similarly, the incidence of severe arthritis (≧index 3) was seen in 60% of the paws of mice injected with pCK, versus only 31% of the paws of mice treated with pCK-sTNFR:Fc 20 days after boosting (P<0.05) (FIG. 4c). However, there was no significant difference in the incidence of arthritis (>index 1) between sTNFR:Fc DNA and the control DNA groups. These results suggested that electroporation-mediated transfer of pCK-sTNFR:Fc could efficiently reduce the incidence of moderate to severe CIA, and also that under the inventive experimental system, beneficial effects of a single electroporation-mediated gene transfer last for a minimum of 18 days following treatment.

### EXAMPLE 3

The Effects of the Electroporation-Mediated Delivery of pCK-sTNFR:Fc on Synovitis and Cartilage Erosion in CIA

The incidence of arthritis in the knee joints was assessed by histological examination. Hematoxylin-eosin staining of knee joint tissues from control mice or mice treated with pCK-sTNFR:Fc. Data are representative of 20 samples. The synovitis could be significantly downgraded as described in the knees of mice treated with pCK-sTNFR:Fc. The score of synovitis was graded as described in Reference Example 6.

Sections stained with hematoxylin and eosin showed that synovial proliferation and inflammatory cell infiltration were significantly decreased in the knee joints of mice treated with sTNFR:Fc, as compared with those of mice injected with the control plasmid (FIG. 5, a and b). Comparison of the histological grades of synovitis between the sTNFR:Fc-treated group and the control group showed that the difference was statistically significant (P<0.05) (FIG. 5, c).

The effects of pCK-sTNFR:Fc on synovitis and cartilage erosion in CIA was analyzed. The score of cartilage destruction was graded as described in Reference Example 6. When pCK-sTNFR:Fc was injected, thinning and hyalinization of the cartilage were also inhibited (FIG. 6). Safranin O-staining of proteoglycan in the cartilage showed that the proteoglycan was well-preserved in the joints of mice treated with sTNFR:Fc, but not in the joints treated with control plasmid DNA (FIG. 6, a and b). A statistically significant difference was found in the severity of cartilage erosion between the sTNFR:Fc-treated group and the control groups (P<0.05) (FIG. 6, c). These results demonstrated that electroporation-mediated delivery of pCK-sTNFR:Fc efficiently reduced the degree of histopathologic changes in the knee joints of CIA mice.

### EXAMPLE 4

The Effects of sTNFR:Fc DNA Transfer on the Cytokine Expression in the Ankle Joints

To further clarify the mechanisms underlying the favorable effects of sTNFR:Fc, the expression levels of inflammatory cytokines were measured by ELISA. Aqueous joint extracts were isolated from ankle joint tissues and analyzed by ELISA. The relative level of vWF was calculated by dividing the mean value of the level of vWF in joint tissue from the experimental mice by the mean value of vWF in joint tissue from the control mice. Values are the mean and SEM from 40 tissue samples per group.

In the sTNFR:Fc-treated mice, the production of IL-1β and IL-12 were reduced to 69 and 16%, respectively, relative to the levels seen in the control vector-treated mice (P<0.01) (FIG. 7), while the levels of IL-17 and wWF remained unchanged in the sTNFR:Fc-treated mice as compared with the control mice. These results suggested that delivery of sTNFR:Fc DNA by electroporation could efficiently reduce the incidence of CIA by modulating the levels of inflammatory cytokines such as IL-1β and IL-12.

While the embodiments of the subject invention have been described and illustrated, it is obvious that various changes and modifications can be made therein without departing from the spirit of the present invention which should be limited only by the scope of the appended claims.

What is claimed is:

1. A pharmaceutical composition for electro-gene therapy of arthritis in a mammal, which comprises a therapeutically effective amount of a DNA encoding soluble p75 TNF (tumor necrosis factor) receptor linked to the Fc portion of human IgG1 (sTNFR:Fc) and a pharmaceutically acceptable carrier.

2. The composition of claim 1, wherein the DNA encoding sTNFR:Fc is contained in an expression vector.

3. The composition of claim 2, wherein the expression vector is pCK-sTNFR:Fc.

4. The composition of claim 1, wherein the mammal is human.

5. The composition of claim 1, which is administered with in vivo electroporation.

6. The composition of claim 1, which is administered to the muscles.

7. A method for electro-gene therapy of arthritis in a mammal, which comprises administering a therapeutically effective amount of a DNA encoding sTNFR:Fc via in vivo electroporation.

8. The method of claim 7, wherein the DNA encoding sTNFR:Fc is contained in an expression vector.

9. The method of claim 8, wherein the expression vector is pCK-sTNFR:Fc.

10. The method of claim 7, wherein the mammal is human.

11. The method of claim 7, wherein the DNA encoding sTNFR:Fc is administered to the muscles.

12. The method of claim 7, wherein the therapeutically effective amount of the DNA encoding sTNFR:Fc ranges from 0.005 to 50 mg/kg body weight.
